(19) United States
(12) Patent Application Publication
(10) Pub. No.: US 2017/0249823 A1
(43) Pub. Date: Aug. 31, 2017

(54) System for Tracking Wellness and Scheduling of Caregiving

(71) Applicant: Manuel A. Espinoza, Irvine, CA (US)

(72) Inventor: Manuel A. Espinoza, Irvine, CA (US)

(21) Appl. No.: 15/594,481

(22) Filed: May 12, 2017

Related U.S. Application Data
(63) Continuation-in-part of application No. 14/856,762, filed on Sep. 17, 2015.
(60) Provisional application No. 62/335,297, filed on May 12, 2016.

(51) Int. Cl. G08B 21/04; H04L 29/08; A61B 5/0205; A61B 5/11; A61B 5/00

(52) U.S. Cl. CPC G08B 21/0446; A61B 5/1117; A61B 5/1112; A61B 5/002; A61B 5/0205; H04L 67/306; A61B 2560/0242; A61B 5/0816

(57) ABSTRACT

Disclosed is a system for tracking wellness and providing caregiving. The system includes a communication device configured to receive health data from at least one personal monitoring device associated with a user and transmit a caregiving notification to a mobile device associated with a caregiver. Further, the system includes a storage device configured to store a plurality of user profiles associated with a plurality of users, store a plurality of caregiver profiles associated with a plurality of caregivers and store the caregiving notification. Further, the system includes a processing device configured to analyze health data received from the at least one personal monitoring device and generate the caregiving notification based on each of a user profile of the user, a caregiver profile of the caregiver and analysis of the health data.

### FIELD OF THE INVENTION

The present invention relates generally to medical systems. More particularly, the present invention relates to a cloud application that will receive data from end users and retrieve environmental data from online media sources and to handle scheduling of caregivers as well as scheduling of care activities.

### BACKGROUND OF THE INVENTION

Currently, People go in for a check-up yearly. When they see their doctor, they have no idea why health problems arise/exist (problems like: dehydration, lack of sleep, fatigue, weight loss/gain, general illness). Further, people do not know when these different types of illness happen.

Further, when people have issues, it could be non-medical related which means the person may not necessarily need a medical professional and the issue could be resolved with the help of a family member. Moreover, when people have issues, they may not even be aware of it and would not know who to go to for help.

Moreover, when people fall, it could be due to various reasons such as, blood pressure issues, stroke, heart attack, heat exhaustion etc. More importantly, when they fall, they're undetected/unreported in most cases.

Furthermore, the use of pendants can also cause additional problems, like the pendant catching on a doorknob, choking the user. Further, when the fall happens, there is lack of information (information which would include heart rate, location, etc.

There are many devices that track and monitor the user's vital information, fall detection and alerting. The issues is that these functions are not integrated into a single devices and require users to have many devices to achieve the desired wellness tracking and monitoring goals. Problems with many devices may include short battery life, short signal range, size of the device, comfort of the user, geolocation accuracy, cell phone service not being available, contact availability, reachability of an SOS button and the user forgetting to wear an alert device around the home.

Another common problem is cost. People often don't want to pay for a monitoring solution if the majority of the time the call center will simply contact a family member to help the user.

It is therefore an objective of the present invention to introduce a system that users can utilize to overcome one or more of the aforementioned problems.

### SUMMARY

Disclosed is a system for tracking wellness and providing caregiving. The system includes a communication device configured to receive health data from at least one personal monitoring device associated with a user and transmit a caregiving notification to a mobile device associated with a caregiver. Further, the system includes a storage device configured to store a plurality of user profiles associated with a plurality of users, store a plurality of caregiver profiles associated with a plurality of caregivers, and store the caregiving notification. Further, the system includes a processing device configured to analyze health data received from the at least one personal monitoring device. The system will then generate the caregiving notification based on each of a user profile of the user, a caregiver profile of the caregiver and analysis of the health data.

In accordance with some embodiments, a cloud application (app) is disclosed. The cloud application handles scheduling of caregivers as well as scheduling of care activities. The cloud application will be able to take care of hundreds of agencies, and thousands of care.

Further, the cloud application may communicate with a mobile application installed on a mobile client. The mobile application provides agencies and family members visibility to track the whereabouts and activities of caregivers. It will also allow family members to participate as caregivers.

In accordance with some embodiments, a mobile application is disclosed. The mobile application allows for tracking and scheduling a plurality of registered health care workers for caregiving sessions.

In accordance with some embodiments, the disclosed system has the ability to provide a remote work force on demand by the system/Cloud app.

In accordance with some embodiments, the disclosed system tracks the wellness of users and dispatches the remote work force to care for users living at home and to administer care and medication.

### BRIEF DESCRIPTION OF THE DRAWINGS

FIG. 1 illustrates a block diagram of a system for tracking wellness and providing caregiving in accordance with some embodiments.

FIG. 2 illustrates a block diagram illustrating an intercom device installed at a premises of a user in accordance with some embodiments.

FIG. 3 illustrates a block diagram of an intercom device in accordance with some embodiments.

FIG. 4 is a flow diagram illustrating various services provided by a smartwatch in accordance with some embodiments.

FIG. 5 is a flowchart illustrating communication by the messaging API in according with some embodiments.

FIG. 6 is a flowchart illustrating the operation of proximity service in according with some embodiments.

FIG. 7 is a flowchart illustrating the operation of the fall detector in according with some embodiments.

FIG. 8 is a flowchart illustrating the operation of the signal collector in according with some embodiments.

FIG. 9 is a flowchart illustrating GPS tracking in according with some embodiments.

FIG. 10 is a flowchart illustrating enabling safe space service in according with some embodiments.

### DETAILED DESCRIPTION OF THE INVENTION

All descriptions are for the purpose of showing selected versions of the present invention and are not intended to limit the scope of the present invention.

Non-limiting and non-exhaustive embodiments of the present invention are described with reference to the preceding figures, wherein like reference numerals refer to like parts throughout the various views unless otherwise precisely specified.

In the description herein, general details of the present invention are provided in flow diagrams to provide a general understanding of the programming methods that will assist in an understanding of embodiments of the present invention. One skilled in the relevant art of programming will recognize, however, that the present invention can be practiced without one or more specific details, or in other programming methods. Referenced throughout this specification to “one embodiment” or “an embodiment” means that a particular feature, structure or characteristic described in connection with the embodiment is included in at least one embodiment of the present invention. Thus, the appearance of the phrases “in one embodiment” or “in an embodiment” in places throughout this specification are not necessarily all referring to the same embodiment. Furthermore, the particular features, structures, or characteristics may be combined in any suitable manner in one or more embodiments.

### Overview

To monitor the health of the client, the cloud app will be able to support personal monitoring devices that will track activity using GPS, pedometers, altimeters, skin temperature, heart rate, and will have fall detection. All the data gathered will be transferred to the cloud application and stored and processed by the system using work flows.

The work flows will send alerts to the care staff and user's family members. The cloud application will allow personal profiles to be developed for each user. Each profile will contain the following: doctor's notes, medication, medication schedule, coverage hours required, and any additional special notes. Each profile will have historical notes entered by the caregiver, historical medication, and photos of any injuries, and/or anything else that might be of interest to the user. In the invention's preferred embodiment, the cloud application will be compliant with HIPAA (Health Insurance Portability and Accountability Act).

Herein follows a list of the present invention's constituent components and processes. In the present invention, a caregiver tracking and scheduling system comprises of: a set of caregiver mobile app processes, a set of docking station sub-processes, a set of processes for intercoms, a set of wearable device processes, and a set of acknowledge-of-alarm processes. There is a process for comparative analysis of current data vs historical data, and the delta from real-time data vs normal baseline readings. One example may be checking for a sudden drop in heart rate; if the heart rate is 20% below the average, for example, no analysis is required for a work flow to be triggered. All workflows may result in a call center reaching out to person who is monitored or to a caregiver.

The first major component/process of the present invention is the set of caregiver mobile app processes. In the invention's preferred embodiment, users who are part of the caretaker work force will have access to the present invention's mobile app. In the invention's preferred embodiment, this app will be compatible with both Android and Apple devices. Alternative or future embodiments may have varying levels of compatibility with different platforms.

Herein follow the major processes of the caregiver app. The app will be used to check availability of the caretaker work force. The app will be used to communicate to a managing agency so work can be booked. The app will allow the caretaker work force to be able to send exceptions when they cannot report for work. The app will be GPS-enabled to track locations of the caretaker work force and better manage time cards. The app will provide notifications to the caretaker work force in administering any medication, special care notes, such as “turn user to the other side,” and see special notes from other caregivers, or from other family members. The app will also allow the caregiver work force to put updates, i.e. put notes, upload photos, and input free text messages that would be used for the care of the user.

Herein follow a list of the functions and web-based user interface features in the invention's preferred embodiment. Notably, the fields included may vary in alternative and future embodiments.

The first component is the Active Caretakers report. This report is a list of active caretakers that are currently on-shift. This report may include the following fields: Caretaker's Profile, User's Name, Caretaker's Skills, Status of Mobile, i.e., ON/OFF. This process may include Search Functionality.

The next component is the set of User Profiles. This includes the User's Name. The next component is the User Devices. This is a list of monitors assigned to a user. Fields may include: User's Name, Device Manufacturer, Device Type, Device Metrics, Device Status, i.e., ON/OFF, and the time of the Last Update. The next component is the Caretaker Profile. This includes the personal details of the person that would function as Caretakers. Fields may include: Username/Password, First Name, Last name, Date of Birth, Company Name, Home Address: Country, Home Address: State, Home Address: City, Home Address, Home Address: Zip Code, Mobile Number, Email Address, Status of Mobile, i.e., ON/OFF, Skills, Special Notes, etc. The next component is the Caretaker Skills. This is a list of certifications and training types, such as CPR, Physical therapist, nutritionist, emergency response, etc. The fields may include: Skill Name, Skill Description. The next component is the Medication and Staff Assignment. This is the scheduling of user care and medication. This is broken-down into subtypes, including Client Information, Client Medication Schedule, and Shift Request. These are described as follows. The Client Information fields may include: Name, Gender, Account Number, Address, and Phone Number. The next component is the User Profile. This contains the personal details of the user. This is also broken-down into subfields. The first subfield is Client Information. Subfields may include: Name, Gender, Account Number, Address such as View Location, Phone Number, Client Regional Center Care. The next subfield is Emergency Contact. Subfields may include: Name, Phone Number, Address, and Relationship. The next subfield is Health Insurance. Subfields may include: Provider and Policy Number. The next subfield is Required Skills. The next subfield is Notes. Subfields may include: Allergies, Medication Notes, and Special Notes. The next major component/process of the present invention is the set of docking station processes. The processes are as follows: The Devices will be able to sit for charging on a docking station. In the invention's preferred embodiment, the docking station will be able to charge two devices. In alternative or future embodiments of the invention, the docking station may charge fewer or more devices.

The docking station will be able to connect via Optics, RJ11 for PSTN. Alternative or future embodiments of the invention may utilize different connection methods.

The docking station will be able to connect via RJ45 to the Internet, to support remote access and webRTC, or similar technologies. The docking station will have 3G/4G support for voice back-up and remote access. Alternative or future embodiments of the invention may utilize other forms of communications.

Notably, the present invention supports a built-in speaker. In the invention's preferred embodiment, this is enabled for two-way communication, and may be accompanied with a microphone. The microphone may be voice-recognition capable. The speaker will play an alarm when a fall is detected. Logs will be accessible remotely and used to create analytics. In the invention's preferred embodiment, there is a 2.4 GHz antenna to passively monitor this band, to detect Wi-Fi and Bluetooth devices, such as but not limited to intercom devices.

Next, the docking station will include an industry-standard CPU, memory, and touch screen LCD panel to process logs, as well as process device location behavior alerts. For example, if the intercom system detects the user normally spends 15 minutes in the toilet, and the device is in the toilet for 60 minutes, a message will be sent to the user's wearable device.

It is important to emphasize, the wearable device itself will not do the detection in the invention's preferred embodiment. The intercoms will perform the detection, and send the information to the docking station. Each intercom device will have a passive antenna that will detect devices based on proximity to it, i.e., signal strength from Bluetooth to intercom. If an intercom is placed in the toilet, when someone with a Bluetooth device is in the toilet, the signal strength will be at its strongest due to proximity.

In the aforementioned situation, if there is no response, an escalation path of alerts and notifications will be followed.

The next major component/process of the present invention is the set of processes for intercoms. The processes are as follows. Intercoms include two-way audio buttons. These are to reach other intercoms or docking station. The intercoms will include an SOS button. When pressed, a call will be placed to the call center. The docking station will bridge the conference intercom with the call center via SIP/PSTN, 3G/4G/5G, or other communications methods. The intercoms will also include a 2.4 GHz antenna to passively monitor this band for Wi-Fi and Bluetooth devices. Alternative or future embodiments may utilize different ranges. Wi-Fi and Bluetooth devices occupying the 2.4 GHz spectrum will be monitored for device ID and signal strength relative to each intercom. The next intercom process is location assignation. Intercoms will be assigned a location. For example, this may be “kitchen, “bedroom,” etc. In the invention's preferred embodiment, the process will use signal strength relative to the intercom as a means to determine the location of wearable devices. The next major component/process of the present invention is the set of wearable device processes. This is the set of processes the device will possess in the invention's preferred embodiment. The wearable device may be a mobile device similar to a pendant, wristband, etc. The processes are as follows. The wearable device includes fall detection; one method of implementation is using a The fall detector may include an accelerometer, a breathing rate sensor and a heart rate sensor, wherein the falling may be detected based on readings from each of the accelerometer, the breathing rate sensor and the heart rate sensor followed by a period of no movement. This implies the user has likely fallen. The wearable device will send a call out to the caregivers or other responsible parties, like an alert center. This occurs as follows.

The fall detection alarm will send a flag signal to the docking station, activating the docking station to announce that someone has fallen on the speaker. The device will then play an alarm sound, and send a message to the user's family members, caregivers and call center. If the alarm is not acknowledged within 180 seconds on the docking station, a help alert will be triggered on the wearable device. Alternative or future embodiments may utilize a different duration.

There may be related battery-saving features. In the event the user becomes unconscious in an out-of-the-way location, the audible alert will not sound until it detects searchers in proximity. This is done through industry-standard geo-fencing. The searchers may have the app installed on their own devices. The reason this geo-fencing activation is done is so the device does not beep incessantly while searchers are not around, thus conserving battery power.

The next process is indoor location tracking using industry-standard Bluetooth; this is used to track the position of the Bluetooth relative to intercom devices. The SOS button may be the same button from the aforementioned set of processes for intercoms. In alternative or future embodiments of the invention, this may be a separate button tied into the same function. The reason for the separate function is to provide an alternative trigger in case the first button is not reachable. The SOS will dial the call center via 3G/4G/5G, or other means of communication.

The wearable device in the invention's preferred embodiment is water proof. This may be implemented through industry-standard means. The wearable device will include a two-way audio and phone function.

In the invention's preferred embodiment, the device will have a button on the front side and the SOS button on the back-side. This is a specification of arrangement. Each button on front will be mapped to a speed-dial number so user can easily contact key family and friends within range of the docking station; if out of range of the docking station contact may be made via webRTC. The wearable devices in the present invention's system will send the log file to the docking station.

The next major component/process of the present invention is the set of acknowledge-of-alarm processes. These are as follows. The docking station will have button that can be pressed, this will send a flag to the wearable device if in range. The call center can acknowledge a triggered alarm remotely on the docking station. It can also accomplish this through a networked connection such as TCP/IP. Notably, a family member of the user can also acknowledge an alarm remotely on the docking station, or through TCP/IP. Alternative or future embodiments of the invention may utilize other communications protocols.

Some exemplary use case scenarios in accordance with the present disclosure may be as follows:
a) Tony is currently in Japan. The app will alert him that since he does not eat meat, he cannot eat the seafood in Japan because there is a current alert of contamination.
b) Lisa is going to another island for her weekend getaway. In one of her adventures in the island, she falls. The app will alert family members of the fall.
c) Bob ate at a restaurant with a bad rating. After a few days, Bob experienced weight loss. Upon retrieving date, it was found out that Bob ate at this specific restaurant, using the GPS data. This may enable the system to make analysis that the cause of Bob's weight loss is the bad food that he ate at this restaurant.
d) Mary goes to the mountain to go biking and the app learns about heavy rains and flash floods. When the system detects the fall, the app can determine the severity of the situation and dispatch the appropriate caregiver resource to help Mary.

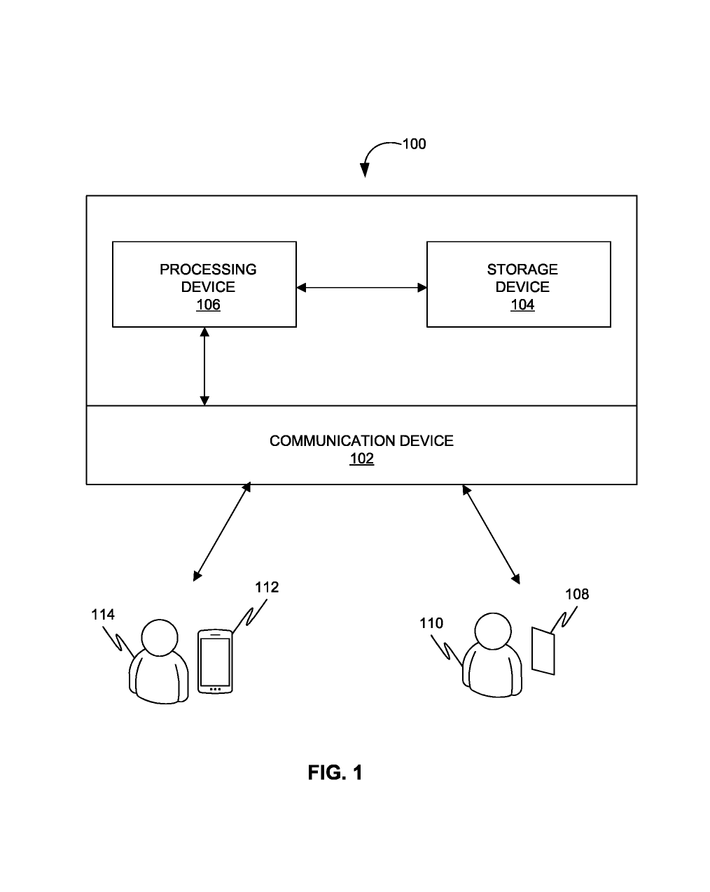

Referring now to figures, FIG. 1 illustrates a block diagram of a system 100 for tracking wellness and providing caregiving in accordance with some embodiments. The system 100 may include a communication device 102, a storage device 104 and a processing device 106.

The communication device 102 may be configured to receive health data from a personal monitoring device 108 associated with a user 110. The user 110 may be a user and the personal monitoring device 108 may be used to track the health status of the user 110. For example, the personal monitoring device 108 may be a wearable device including, but not limited to, a smart watch, a smart ring, smart glasses, smart shoes and/or smart clothing.

In addition, in some embodiments, the communication device 102 may also receive relevant data from online media resources such as CDC (center for disease control), weather.com, yelp, traffic app, information about car accidents, problems with the roads, etc. These online media resources will be localized based on the user's location. Accordingly, in an instance, the system (for example, in cooperation with the cloud app) may schedule or dispatch caregiver resources based on result of the analysis (e.g. big data analysis) on data collected by the wristband and environmental data collected from online media resources. The environmental data collected from online resources may be used by the system to determine the severity and assess the risk.

Other online resources might be traffic apps, which will be able to track car accidents, problems with the roads etc. that might be relevant to people who are traveling in those areas. Accordingly, if a fall is detected, this information would be sent to the caregiver that would be dispatched.

Further, the communication device 102 may be configured to transmit a caregiving notification to a mobile device 112 associated with a caregiver 114. The caregiver 114 may be a doctor, a nurse, a physician assistant, an orderly or other hospital staff, a family member, a friend, a passerby and so on. For example, the mobile device 112 may be a smartphone or a smart tablet. Further, the mobile device 112 may employ an operating system including, but not limited to, Android, iOS, Symbian, Windows Phone and BlackBerry OS.

The caregiving notification may include each of the user profile and a location of the user 110. Further, the caregiving notification may include a caregiver schedule, wherein the caregiver schedule may include each of the user profile and a time corresponding to caregiving. The communication device 102 may be further configured to receive an update from the mobile device 112 corresponding to caregiving of the user 110.

The communication device 102 may communicate with the personal monitoring device 108 and the mobile device 112 via one or both of a wired or a wireless connection. For example, the wireless connection may be established based on one or more communication protocols including, but not limited to, 3G, 4G, Wi-Fi, RFID, ZigBee and Bluetooth.

The storage device 104 may be configured to store multiple user profiles associated with multiple users (such as the user 110). Further, the storage device 104 may be configured to store multiple caregiver profiles associated with multiple caregivers (such as the caregiver 114). Yet further, the storage device 104 may be configured to store the caregiving notification.

The processing device 106 may be configured to analyze health data received from the personal monitoring device 108. Further, the processing device 106 may be configured to generate the caregiving notification based on each of a user profile of the user 110, a caregiver profile of the caregiver 114 and analysis of the health data. The processing device 106 may be further configured to generate an active caregivers report based on the caregiving notification.

According to further embodiments, the communication device 102 may be further configured to receive a status of the mobile device 112. The status may indicate an availability of the caregiver 114, wherein the processing device 106 may be configured to determine the caregiving notification based on the status.

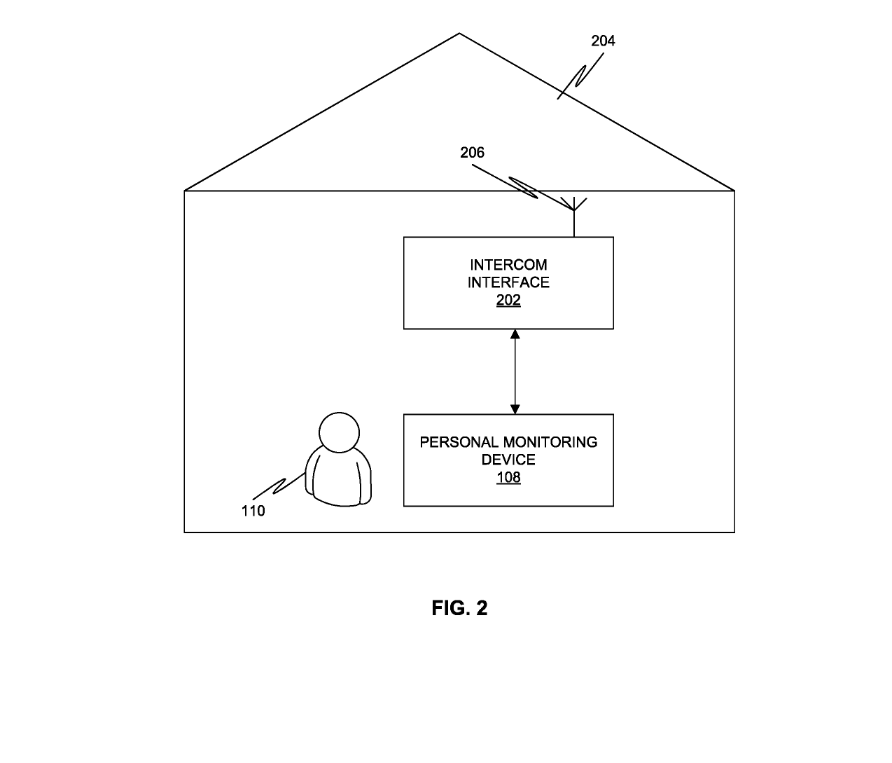

As shown in FIG. 2, the communication device 102 may be further configured to communicate with an intercom device 202 installed at a premises 204 of the user 110. Further, the intercom device 202 is configured to communicate with the personal monitoring device 108. The personal monitoring device 108 may be a wearable device.

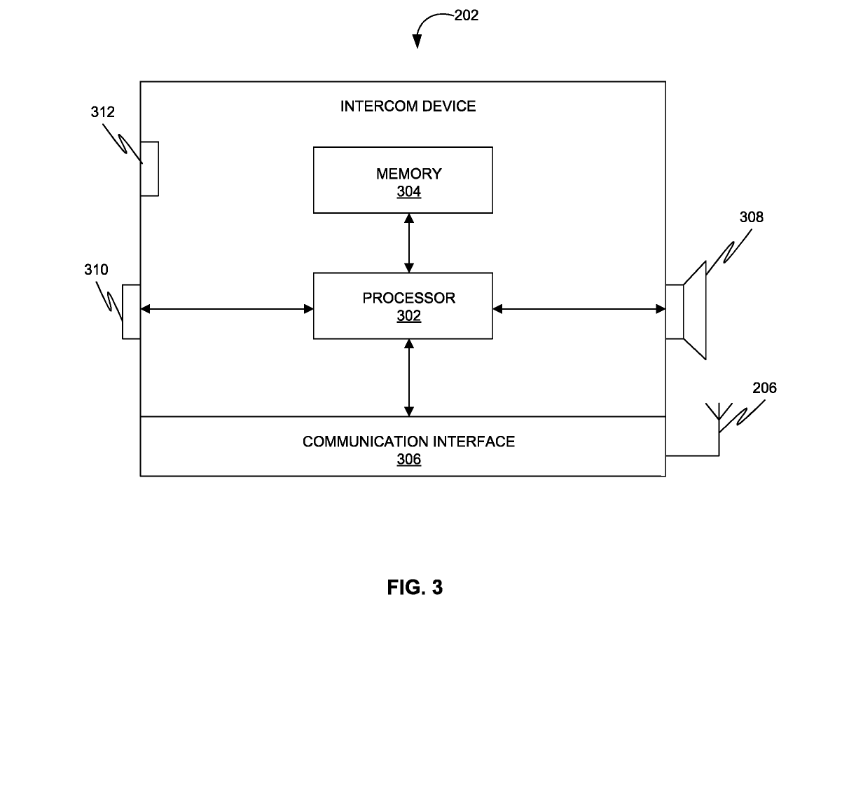

FIG. 3 illustrates a block diagram of the intercom device 202 in accordance with some embodiments. The intercom device 202 includes a processor 302, a memory 304, a communication interface 306, a speaker 308 and a microphone 310. The intercom device 202 further includes a charger 312 configured to provide charging power to one or more mobile devices.

Further, the intercom device 202 may include an antenna 206 configured to detect strength of a signal transmitted by the wearable device 108 worn by the user 110. The strength of the signal indicates a location of the user 110 in relation to the intercom device 202. The intercom device 202 may be installed at a predefined location in the premises 204 of the user 110.

According to some embodiments, the wearable device 108 may include a two-way phone configured to communicate with the communication interface 306 of the intercom device 202. The two-way phone further includes multiple speed dial buttons corresponding to multiple telephones, wherein pressing of a speed dial button establishes a call with a corresponding telephone of the multiple telephones.

According to some embodiments, the wearable device 108 may include an SOS button (not shown). The wearable device 108 may be further configured to communicate an SOS notification to the intercom device 202 based on pressing of the SOS button. The intercom device 202 may be configured to transmit the SOS notification and the communication device 102 may be configured to receive the SOS notification from the intercom device 202.

Further, the intercom device 202 may also include an SOS button. The intercom device 202 may be configured to transmit an SOS notification based on a pressing of the SOS button, and the communication device 102 may be configured to receive the SOS notification from the intercom device 202.

Yet further, the wearable device 108 may include a fall detector configured to detect falling of the user 110. The fall detector may include an accelerometer, a breathing rate sensor and a heart rate sensor, wherein the falling may be detected based on readings from each of the accelerometer, the breathing rate sensor and the heart rate sensor. Further, the intercom device 202 may be further configured to receive a fall detection from the fall detector, wherein the processor 302 may be configured to play an alarm sound using the speaker 308 based on the fall detection. The processor 302 may be further configured to initiate a timer based on receipt of the fall detection, wherein the communication interface 306 may be further configured to transmit a help alert to the system 100 upon expiration of the timer unless an acknowledgement of the alarm sound is received at the intercom device 202. For example, the acknowledgement may be received by pressing of a button comprised in the intercom device 202 or the acknowledgement may be received through the communication interface 306 of the intercom device 202.

The intercom device 202 may be configured to determine presence of the mobile device 112 within a geo-fence associated with the premises 204. The processor 302 of the intercom device 202 may be configured to play the alarm sound based on presence of the mobile device 112.

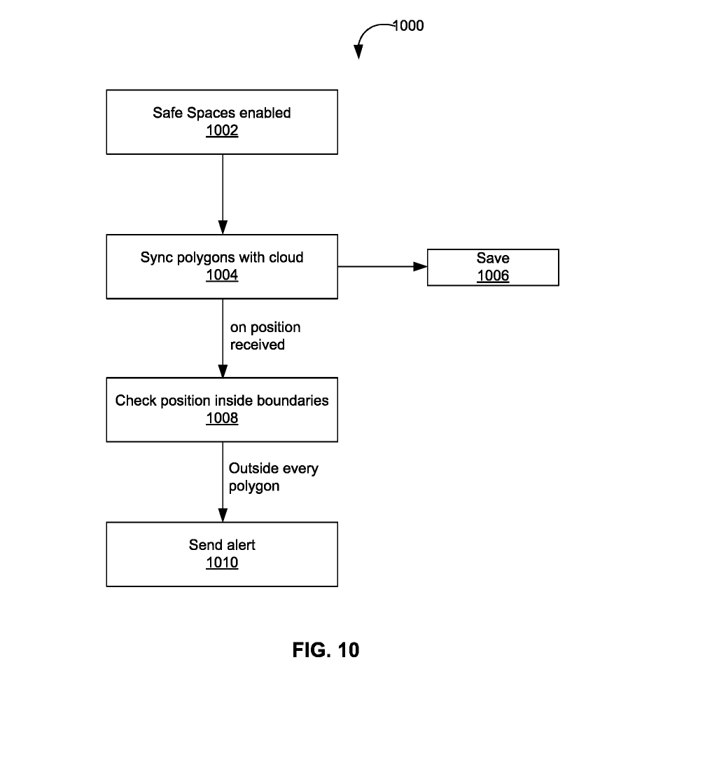

The personal monitoring device 108 may include a location sensor configured to determine a geolocation of the user 110. Accordingly, the processing device 106 may be further configured to generate the caregiver notification further based on the geolocation. Further, the communication device 102 may be configured to receive an indication of one or more safe spaces, wherein the processing device 106 may be further configured to generate the caregiver notification based on the geolocation of the user 110 being outside the one or more safe spaces. This is explained in further detail in conjunction with FIG. 10 below.

According to some embodiments, the system 100 may be implemented in a cloud computing environment. The cloud computing environment may include third party providers including, but not limited to, Microsoft Azure, Amazon EC2, and Google AppEngine. The system 100 receives data from multiple personal monitoring devices (such as personal monitoring device 108) via one or both of a mobile application and REST API calls.

The system 100 may execute a web application configured to enable user registration, handle user profiles, manage alerts, manage access and sensor administration. Administrators can also check and edit user's data. Administrators can also manage permissions of the different user levels, such as guest, user, supervisor, and admin. The administrators can also define new sensors, therefore metrics types.

Further, the web application may be configured to provide a supervisor's dashboard that contains all pending alerts of a supervised group. Further, the web application may be configured to provide a help system, which may be an editable HTML help system, like a user's manual, and an online help with short tips.

A user application may be installed on one or both of a standalone device (such as the intercom device 202) and a personal communication device, such as a smartphone. The user application will allow users to create groups and invite other users into them to share metrics, achievements or to dedicate that group as an emergency group to alert on events. Further, the user application will display collected alerts for the alert group members in near real time by using a notification framework. Users may also have access to an alert history and pending alert view. Further, the user application will allow the users to log historical data that cannot be measured by wearable devices. Moreover, the user application will allow the users to define goals for logged or measured data. When the goal is reached the web application may issue a badge. The user application may also display a map with GPS tracking information. Further, the user application may also display one or more of step count of the user, activities performed by the user and heart rate of the user.

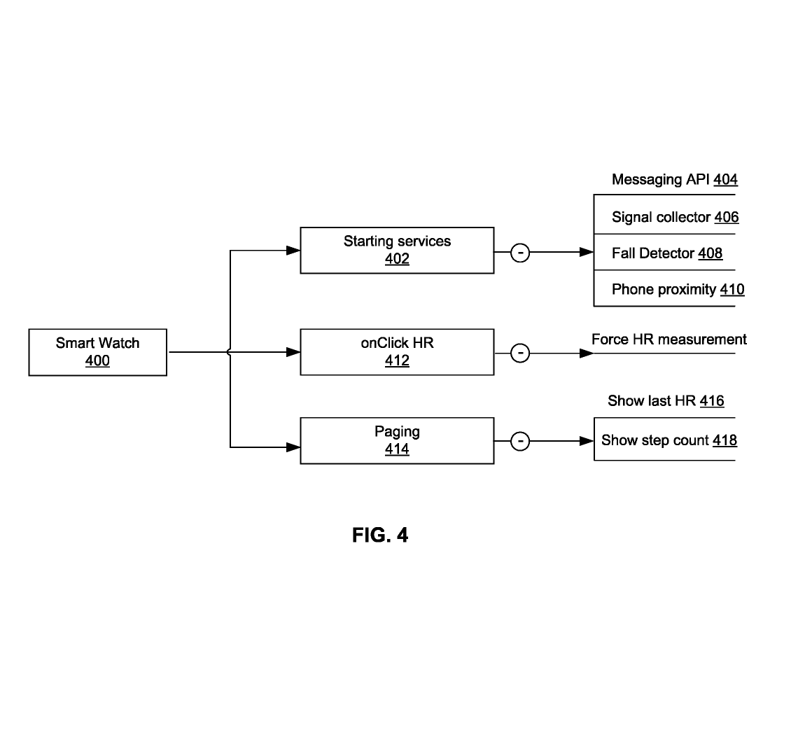

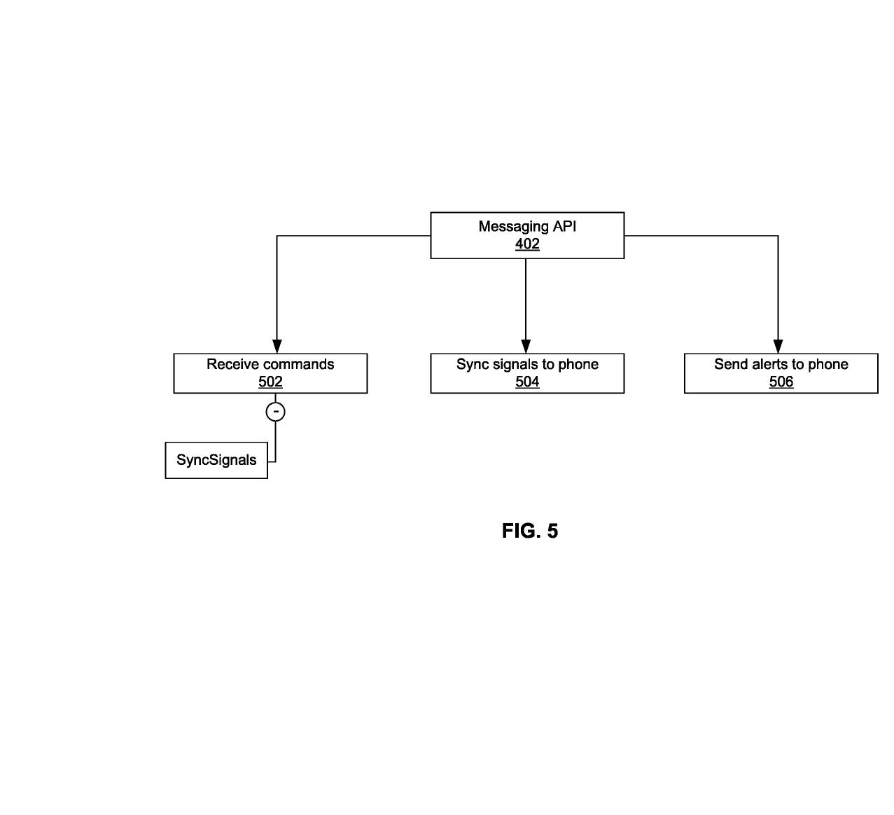

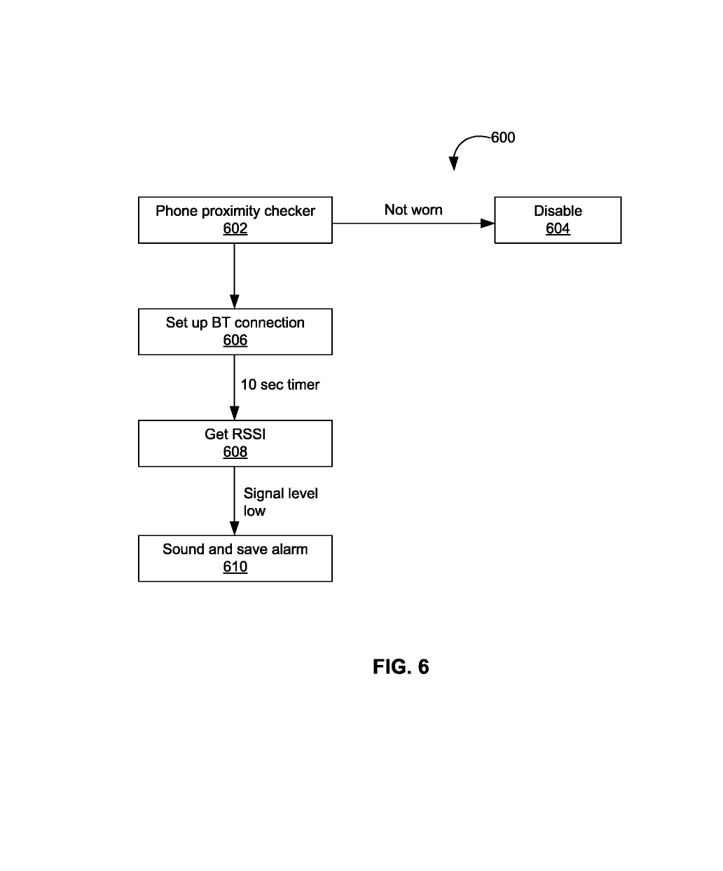

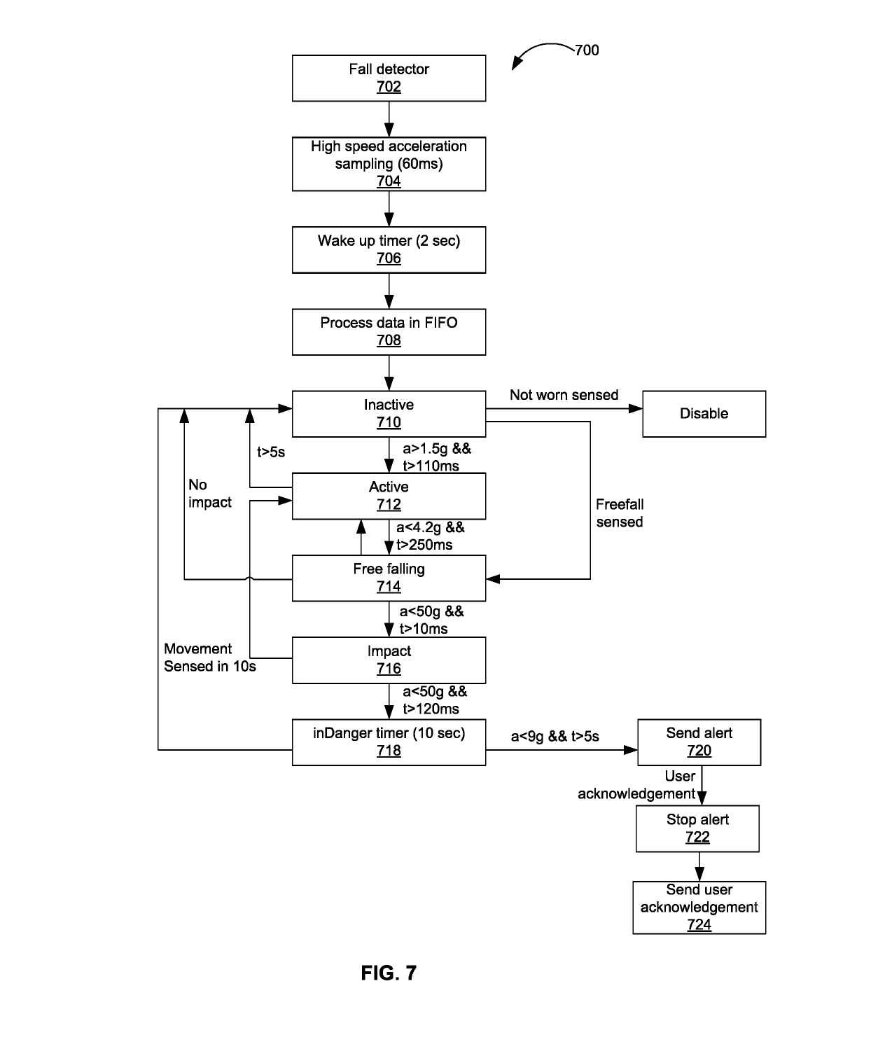

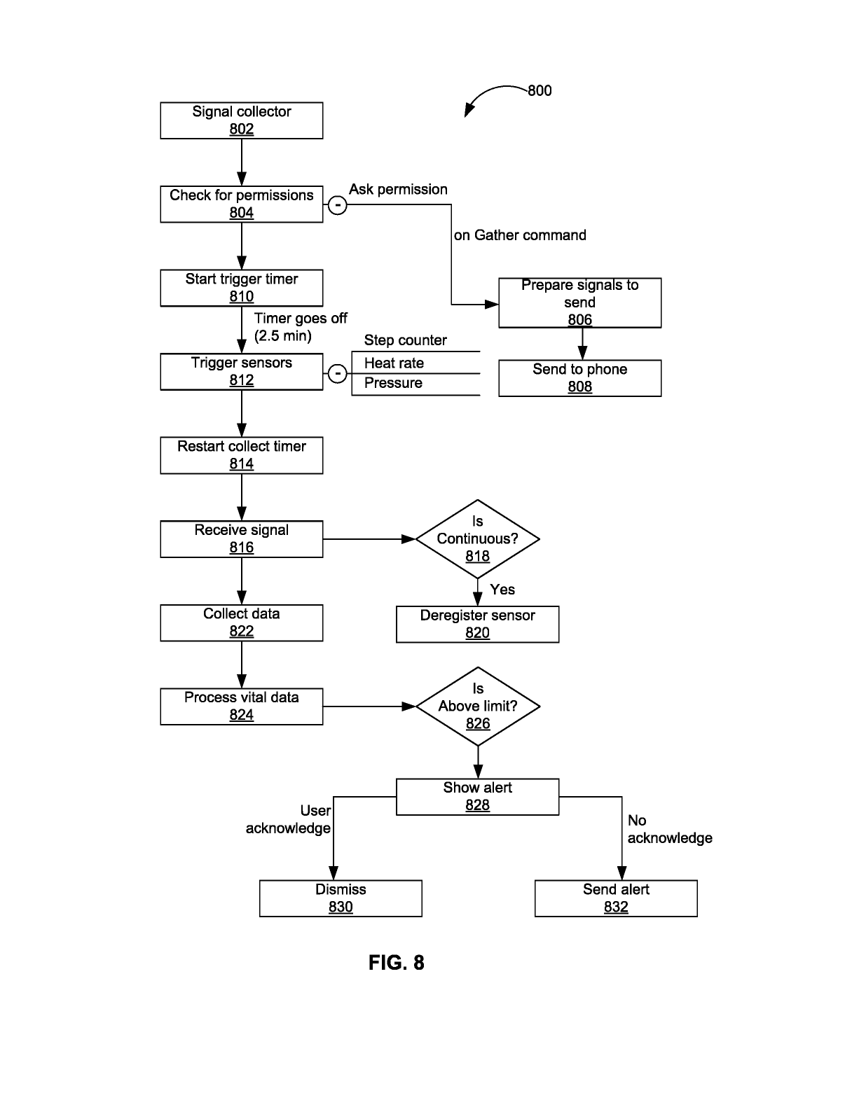

The user application may also allow the user to connect their wearable devices (such as smartwatch, wrist-band, armband, ankle-band, neck-band etc.) to their personal communication device. In some embodiments, the user application may be installed in the wearable devices. FIG. 4 is a flow diagram illustrating various services provided by a smartwatch 400. As shown, when the smartwatch 400 is switched on, starting services are initiated at 402, which include messaging API 404, phone proximity 406, fall detector 408 and signal collector 410. The messaging API 404 is explained in further detail in conjunction with FIG. 5 below. The phone proximity 406 is explained in further detail in conjunction with FIG. 6 below. The fall detector 408 is explained in further detail in conjunction with FIG. 7 below. The signal collector 410 is explained in further detail in conjunction with FIG. 8 below.

Further, the user may check various physiological parameters, such as a heart rate, by interacting with the smartwatch. For example, the user may check heart rate (HR) by clicking appropriate user interface element provided on the smartwatch at 412. Moreover, the smartwatch 400 includes paging capabilities 414, which may be used to show last HR measurement 416 and step count 418.

FIG. 5 is a flowchart illustrating communication by the messaging API 404 in according with some embodiments. The messaging API 404 receives commands at 502, sync signals to the user's smartphone at 504 and sends alerts to the user's smartphone at 506.

FIG. 6 is a flowchart 600 illustrating the operation of proximity service in according with some embodiments. The intercom device 202 may initiate proximity service at 602. If it is determined that the user is not carrying their smartphone or not wearing a wearable device, then the operation disables the proximity service at 604. Otherwise, the intercom device 202 sets up a Bluetooth connection with one or both of the user's smartphone and user's wearable device at 606. Next, the intercom device 202 detects strength of a signal ((Received Signal Strength Indicator) RSSI) transmitted by one or both of the user's smartphone and user's wearable device at 608. If the signal strength is found to be low, the intercom device 202 triggers an alarm at 610.

FIG. 7 is a flowchart 700 illustrating the operation of the fall detector in according with some embodiments. The user's wearable device may include the fall detector. At 702, the fall detector is initiated. The fall detector may include an accelerometer. The sampling rate of the accelerometer may be set at 704. A wake up timer of the fall detector may be set at 2 seconds at 706. The data collected by the fall detector is processed in a First-In-First-Out (FIFO) fashion at 708.

At 710, the fall detector is inactive. If an acceleration greater than 1.5 g is detected for more than 110 ms, then the fall detector becomes active at 712. Further, if an acceleration less than 4.2 g is detected for more than 250 ms, then the fall detector detects a free falling situation at 714. However, if an acceleration less than 4.2 g is not detected for more than 250 ms, then the fall detector goes back to inactive state at 710.

If an acceleration less than 50 g is detected for more than 10 ms, then the fall detector detects an impact at 716. However, if an acceleration less than 50 g is not detected for more than 10 ms, then the fall detector goes back to inactive state at 710.

Thereafter, if an acceleration less than 50 g is detected for more than 120 ms, then an in Danger timer of 10 seconds is initiated at 718. If a user acknowledgment is not received within 10 seconds, then an alert is sent at 720. However, if a user acknowledgment is received within 10 seconds, then the alert is stopped at 722 and the user is informed at 724.

FIG. 8 is a flowchart 800 illustrating the operation of the signal collector in according with some embodiments. At 802, the signal collector is initiated. Then, at 804, the permissions to trigger sensors is checked. If required, the permission is request by preparing signals at 806, which are then sent to the smartphone at 808. If the necessary permissions are available, a trigger timer is started at 810. The timer goes off and the sensors are triggered at 812. Accordingly, one or more of step counter, heart rate and blood pressure are determined. The collect timer is restarted at 814. Thereafter, a signal from a sensor is received at 816. If the signal is found to be continuous at 818, then the corresponding sensor is deregistered at 820. However, if the signal is not continuous, the data is collected at 822 and the vital data is processed at 824. If any data is found to be above limit at 826, then an alert is shown at 828. If an acknowledgment is received from a user, then the alert is dismissed at 830. However, if an acknowledgment is received from a user, then the alert is sent at 832.

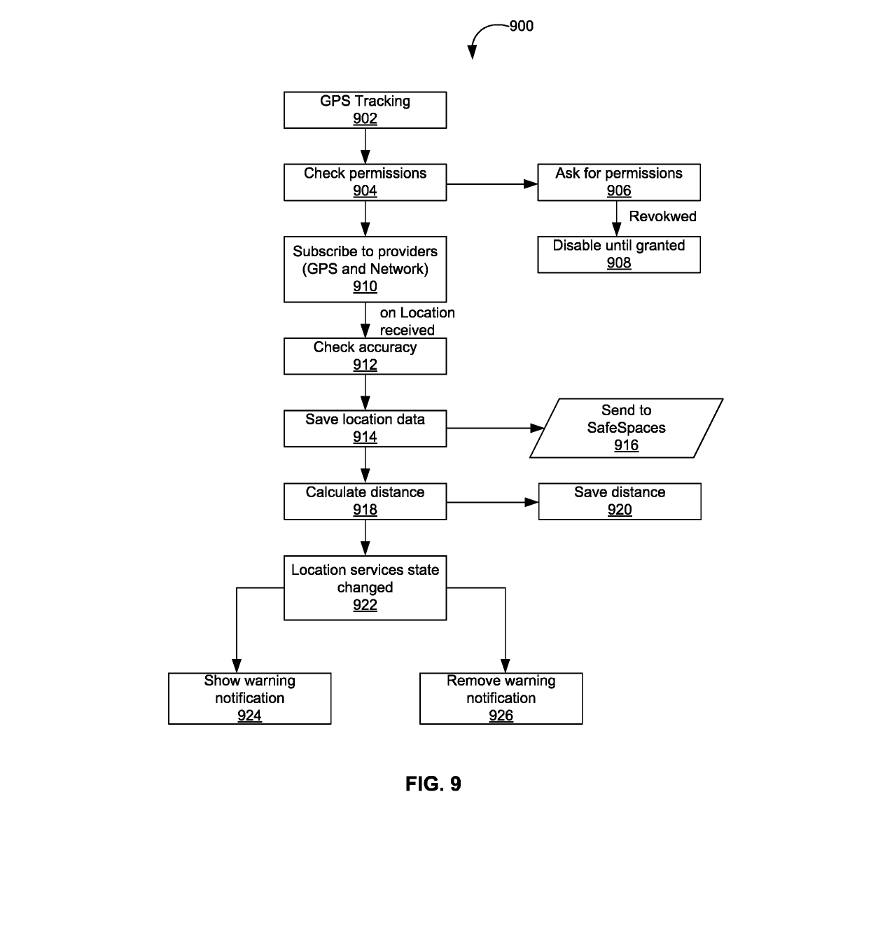

FIG. 9 is a flowchart 900 illustrating GPS tracking in according with some embodiments. At 902, GPS tracking is enabled. At 904, it is checked if the necessary permissions to conduct GPS tracking are available. If the permissions are not available, then request for permission is sent at 906 and the GPS tracking is disabled until permissions are granted at 908. However, if the permissions are available, then subscription to providers is obtained at 910 and accuracy is checked at 912. Further, the location data is saved at 914 and the data is sent to safe spaces service at 916. This is explained in further detail in conjunction with FIG. 10 below.

Next, the distance is calculated at 918 and the distance is saved at 920. Then based on the state changes determined at 922, a warning notification is either shown at 924 or dismissed at 926.

FIG. 10 is a flowchart 1000 illustrating enabling safe space service in according with some embodiments. At 1002, safe spaces service is enabled. Then, the polygons defining safe spaces are synced with the cloud at 1004, which are then saved at 1006. Thereafter, the user position is tracked. If the position of the user is found to be within boundaries of polygons at 1008, then no action is taken. However, if the position of the user is found to be out of all polygons, then an alert is sent 1010.

### Exemplary Embodiments

According to some embodiments of the present disclosure, a system for tracking wellness and providing caregiving is provided. The system may include a communication device configured to receive health data from at least one personal monitoring device associated with a user. Further, the communication device may be configured to transmit a caregiving notification to a mobile device associated with a caregiver. Further, the system may include a storage device configured to store a plurality of user profiles associated with a plurality of users. Further, the storage device may be configured to store a plurality of caregiver profiles associated with a plurality of caregivers. Further, the storage device may be configured to store the caregiving notification. Further, the system may include a processing device configured to: analyze health data received from the at least one personal monitoring device. Further, the processing device may be configured to generate the caregiving notification based on each of a user profile of the user, a caregiver profile of the caregiver and analysis of the health data.

In some embodiments, the caregiving notification may include each of the user profile and a location of the user.

In some embodiments, the caregiving notification may include a caregiver schedule. Further, the caregiver schedule may include each of the user profile and a time corresponding to caregiving.

In some embodiments, the communication device may be configured to receive a status of the mobile device. Further, the status indicates an availability of the caregiver. Further, the processing device may be configured to determine the caregiving notification based on the status.

In some embodiments, the communication device may be further configured to receive an update from the mobile device. Further, the update corresponds to caregiving of the user.

In some embodiments, the processing device may be further configured to generate an active caregivers report based on the caregiving notification.

In some embodiments, the communication device may be further configured to communicate with at least one intercom device installed at a premises of the user. Further, an intercom device may include a processor, a memory, a communication interface, a speaker and a microphone.

In some embodiments, the intercom device may be configured to communicate with the at least one personal monitoring device. Further, the at least one personal monitoring device may include at least one wearable device.

In some embodiments, the intercom device may include an antenna configured to detect strength of a signal transmitted by the at least one wearable device worn by the user. Further, the strength of the signal indicates a location of the user in relation to the intercom device. Further, the intercom device may be installed at a predefined location in the premises of the user.

In some embodiments, the at least one wearable device may include a two-way phone configured to communicate with the communication interface of the intercom device. Further, the two-way phone further may include a plurality of speed dial buttons corresponding to a plurality of telephones. Further, pressing of a speed dial button establishes a call with a corresponding telephone of the plurality of telephones.

In some embodiments, the at least one wearable device may include an SOS button. Further, the at least one wearable device may be further configured to communicate an SOS notification to the intercom device based on pressing of the SOS button. Further, the intercom device may be configured to transmit the SOS notification. Further, the communication device of the system may be configured to receive the SOS notification from the intercom device.

In some embodiments, the intercom device may include an SOS button. Further, the intercom device may be configured to transmit an SOS notification based on a pressing of the SOS button. Further, the communication device of the system may be configured to receive the SOS notification from the intercom device.

In some embodiments, the at least one wearable device may include a fall detector configured to detect falling of the user.

In some embodiments, the fall detector may include an accelerometer, a breathing rate sensor and a heart rate sensor. Further, the falling may be detected based on readings from each of the accelerometer, the breathing rate sensor and the heart rate sensor.

In some embodiments, the intercom device may be further configured to receive a fall detection from the fall detector. Further, the processor of the intercom device may be configured to play an alarm sound using the speaker based on the fall detection. Further, the processor may be further configured to initiate a timer based on receipt of the fall detection. Further, the communication interface of the intercom device may be further configured to transmit a help alert to the system upon expiration of the timer unless an acknowledgement of the alarm sound may be received at the intercom device.

In some embodiments, the acknowledgement may be received by pressing of a button comprised in the intercom device.

In some embodiments, the acknowledgement may be received through the communication interface of the intercom device.

In some embodiments, the intercom device may be configured to determine presence of the mobile device within a geo-fence associated with the premises. Further, the processor of the intercom device may be configured to play the alarm sound based on presence of the mobile device.

In some embodiments, the at least one personal monitoring device may include a location sensor configured to determine a geolocation of the user. Further, the processing device may be further configured to generate the caregiver notification further based on the geolocation.

In some embodiments, the communication device may be configured to receive an indication of at least one safe space. Further, the processing device may be further configured to generate the caregiver notification based on the geolocation of the user being outside the at least one safe space.

Although the invention has been explained in relation to its preferred embodiment, it is understood that many other possible modifications and variations can be made without departing from the spirit and scope of the invention as herein described.

I claim:

1. A system for tracking wellness and scheduling caregiving, the system comprising:
a communication device configured to:
receive health data from at least one personal monitoring device associated with a user;
transmit a caregiving notification to a mobile device associated with a caregiver;
a storage device configured to:
store a plurality of user profiles associated with a plurality of users;
store a plurality of caregiver profiles associated with a plurality of caregivers;
store the caregiving notification; and
a processing device configured to:
analyze health data received from the at least one personal monitoring device;
generate the caregiving notification based on each of a user profile of the user, a caregiver profile of the caregiver and analysis of the health data.

2. The system of claim 1, wherein the caregiving notification comprises each of the user profile and a location of the user.

3. The system of claim 1, wherein the communication device is further configured to receive environmental data from at least one online source, wherein the environmental data corresponds to a location of the user, wherein the processing device is further configured to generate the caregiving notification based further on an analysis of the environmental data.

4. The system of claim 1, wherein the communication device is configured to receive a status of the mobile device, wherein the status indicates an availability of the caregiver, wherein the processing device is configured to determine the caregiving notification based on the status.

5. The system of claim 1, wherein the communication device is further configured to receive an update from the mobile device, wherein the update corresponds to caregiving of the user.

6. The system of claim 1, wherein the processing device is further configured to generate an active caregivers report based on the caregiving notification.

7. The system of claim 1, wherein the communication device is further configured to communicate with at least one intercom device installed at a premises of the user, wherein an intercom device comprises a processor, a memory, a communication interface, a speaker and a microphone.

8. The system of claim 7, wherein the intercom device is configured to communicate with the at least one personal monitoring device, wherein the at least one personal monitoring device comprises at least one wearable device.

9. The system of claim 8, wherein the intercom device comprises an antenna configured to detect strength of a signal transmitted by the at least one wearable device worn by the user, wherein the strength of the signal indicates a location of the user in relation to the intercom device, wherein the intercom device is installed at a predefined location in the premises of the user.

10. The system of claim 9, wherein the at least one wearable device comprises a two-way phone configured to communicate with the communication interface of the intercom device, wherein the two-way phone further comprises a plurality of speed dial buttons corresponding to a plurality of telephones, wherein pressing of a speed dial button establishes a call with a corresponding telephone of the plurality of telephones.

11. The system of claim 9, wherein the at least one wearable device comprises an SOS button, wherein the at least one wearable device is further configured to communicate an SOS notification to the intercom device based on pressing of the SOS button, wherein the intercom device is configured to transmit the SOS notification, wherein the communication device of the system is configured to receive the SOS notification from the intercom device.

12. The system of claim 9, wherein the intercom device comprises an SOS button, wherein the intercom device is configured to transmit an SOS notification based on a pressing of the SOS button, wherein the communication device of the system is configured to receive the SOS notification from the intercom device.

13. The system of claim 9 wherein the at least one wearable device comprises a fall detector configured to detect falling of the user.

14. The system of claim 13, wherein the fall detector comprises an accelerometer, a breathing rate sensor and a heart rate sensor, wherein the falling is detected based on readings from each of the accelerometer, the breathing rate sensor and the heart rate sensor.

15. The system of claim 13, wherein the intercom device is further configured to receive a fall detection from the fall detector, wherein the processor of the intercom device is configured to play an alarm sound using the speaker based on the fall detection, wherein the processor is further configured to initiate a timer based on receipt of the fall detection, wherein the communication interface of the intercom device is further configured to transmit a help alert to the system upon expiration of the timer unless an acknowledgement of the alarm sound is received at the intercom device.

16. The system of claim 15, wherein the acknowledgement is received by pressing of a button comprised in the intercom device.

17. The system of claim 15, wherein the acknowledgement is received through the communication interface of the intercom device.

18. The system of claim 1, wherein the intercom device is configured to determine presence of the mobile device within a geo-fence associated with the premises, wherein the processor of the intercom device is configured to play the alarm sound based on presence of the mobile device.

19. The system of claim 1, wherein the at least one personal monitoring device comprises a location sensor configured to determine a geolocation of the user, wherein the processing device is further configured to generate the caregiver notification further based on the geolocation.

20. The system of claim 19, wherein the communication device is configured to receive an indication of at least one safe space, wherein the processing device is further configured to generate the caregiver notification based on the geolocation of the user being outside the at least one safe space.
